(19) United States
(12) Patent Application Publication
(10) Pub. No.: US 2022/0395010 A1
(43) Pub. Date: Dec. 15, 2022

(54) PROCESS FOR THE PRODUCTION OF TAMARIND EXTRACT

(71) Applicants: Giuseppe CERFOGLI, Nonantola (MO) (IT); Mauricio De Jesus PICHIN, Carpi (MO) (IT); Jesus Rafael RODRIGUEZ AMADO, Manaus (BR); Julio Cesar ESCALONA, Campobasso (CB) (IT); Industria Tecnologica Emiliana S.r.l., Nonantola (MO) (IT)

(72) Inventors: Giuseppe CERFOGLI, Nonantola (MO) (IT); Mauricio De Jesus PICHIN, Carpi (MO) (IT); Jesus Rafael RODRIGUEZ AMADO, Manaus (BR); Julio Cesar ESCALONA, Campobasso (CB) (IT)

(73) Assignees: Giuseppe CERFOGLI, Nonantola (MO) (IT); Mauricio De Jesus PICHIN, Carpi (MO) (IT); Jesus Rafael RODRIGUEZ AMADO, Manaus (BR); Julio Cesar ESCALONA, Campobasso (CB) (IT); Industria Tecnologica Emiliana S.r.l., Nonantola (MO) (IT)

(21) Appl. No.: 17/770,048

(22) PCT Filed: Oct. 23, 2020
(86) PCT No.: PCT/IB2020/059970

§ 371 Date: Apr. 19, 2022

(30) Foreign Application Priority Data
Oct. 24, 2019 (IT) 102019000019727

(51) Int. Cl. A23L 33/105; A61K 36/48

(52) U.S. Cl. CPC A23L 33/105; A61K 36/48

(57) ABSTRACT

The process for the production of Tamarind extract comprises:
a) a supply phase of a vegetable substrate comprising leaves of Tamarindus indica L.;
b) a drying phase of the leaves of Tamarindus indica L.;
c) a grinding phase of the leaves of Tamarindus indica L.;
d) a maceration phase of the leaves of Tamarindus indica L. in an organic solvent
to obtain a mixture;
e) an extraction phase of the Tamarind extract from the mixture obtained in d) to obtain a solution; and
f) a concentration phase of the Tamarind extract.

### TECHNICAL FIELD

The present invention relates to a process for the production of Tamarind extract.

### BACKGROUND ART

To date, liver disease is one of the leading causes of death in the world.

However, the drugs currently used to treat this disease are not sufficient to meet clinical needs.

Generally, liver disease shows itself with changes in bilirubin levels and increases in transaminase levels.

In addition to this there are alterations in lipid metabolism and cholesterol and LDL levels which, by producing lipid peroxidation, determine the formation of many reactive oxygen species.

These species alter liver function, thus modifying the permeability of the liver membrane with consequences that can be also fatal for patients.

Among the most common liver lesions is the acute viral hepatitis A, B, C, D and E, which causes jaundice and liver inflammation also widespread at systemic level.

In detail, hepatitis B does not produce symptoms but produces alterations in haematological and biochemical parameters. In parallel, hepatitis C produces persistent inflammation in the liver that causes a progressive necrotic lesion with risk of cirrhosis and death.

The drugs currently on the market have many drawbacks, including the fact that they have little stability over time and considerably high production costs.

In the face of these diseases, the need is particularly felt to develop new pharmaceutical formulations with hepatoprotective and antioxidant effects with production costs significantly lower than the drugs currently on the market.

### DESCRIPTION OF THE INVENTION

The main aim of the present invention is to devise a process for the production of Tamarind extract that allows obtaining an extract that is stable over time and can be used in pharmaceutical formulations.

Within this aim, one object of the present invention is to devise a process for the production of Tamarind extract that allows obtaining an extract with hepatoprotective and antioxidant function.

Another object of the present invention is to devise a process for the production of Tamarind extract that allows overcoming the above mentioned drawbacks of the prior art in a simple, rational, easy, effective to use and low cost solution.

The above mentioned objects are achieved by the present process for the production of Tamarind extract, having the characteristics of claim 1.

### BRIEF DESCRIPTION OF THE DRAWINGS

Other characteristics and advantages of the present invention will be more evident from the description of a preferred, but not exclusive, embodiment of a process for the production of Tamarind extract, illustrated by way of an indicative, yet non-limiting example, in the attached tables of drawings wherein:

FIG. 1 is a comparative column chart of the concentration of malondialdehyde, superoxide dismutase, catalase and glutathione production in the liver of the experimental models during the testing of the antioxidant activity of the concentrated tamarind extract (EMT) tablets.

### EMBODIMENTS OF THE INVENTION

The present invention relates to a process for the production of Tamarind extract.

It is specified that, unless otherwise indicated, the term “approximately”, when used in association with a numerical value, means that the value should be considered as including the error (e.g., standard error) associated with the obtaining or with the derivation thereof.

In addition, unless otherwise indicated, the terms “to treat” and “treatment” relate to an action that occurs when a patient is suffering from the specified disease or disorder, which reduces the severity of the disease or disorder, or delays or slows down the progression of the disease or disorder.

Finally, unless otherwise specified, the term “to comprise” has the same meaning as the term “to comprise, but not as a limitation”, and the term “to include” has the same meaning as the term “to include, but not as a limitation”. Similarly, the term “such as, e.g.” has the same meaning as “such as, e.g., but not as a limitation”.

The process comprises a supply phase of a vegetable substrate comprising leaves of Tamarindus indica L.

Following this, the process comprises a drying phase of the leaves of Tamarindus indica L.

The drying phase is carried out by exposure of the leaves of Tamarindus indica L. to a heat source.

Preferably, the drying phase comprises a shielding step of the leaves of Tamarindus indica L.

In this regard, it is worth specifying that the shielding step comprises arranging a protective element on top of the Tamarind leaves in order to avoid the direct exposure of the latter to the heat source.

Advantageously, the drying phase allows obtaining leaves of Tamarindus indica L. with a moisture content comprised between 8% and 12%.

It cannot however be ruled out from the scope of the present discussion that the drying phase is carried out by means of: static bed dryers, fluid bed dryers, vacuum dryers, microwave dryers, spray dryers or freeze dryers.

Following this, the process comprises a grinding phase of the leaves of Tamarindus indica L.

The grinding phase is carried out using grinding devices of the mechanical type, such as e.g. a mortar.

It is specified that in the present discussion the term “grinding” is defined as the size reduction of coarse pieces into smaller particles thus allowing the increase in the specific surface area of each fragment of the leaves of Tamarindus indica L. and, in parallel, the increase in the number of fragments per unit of mass.

For this purpose, the grinding phase comprises a sifting step of the leaves fragments of Tamarindus indica L. to obtain fragments ranging from 2.5 mm to 10 mm.

Preferably, the above leaves fragments of Tamarindus indica L. have an average size of approximately 5 mm.

At this point, the process comprises a wetting phase of the leaves fragments of Tamarindus indica L. for a period ranging from 1 hour to 5 hours.

Preferably, the wetting phase lasts approximately 3 hours.

The wetting phase is carried out using an organic solvent comprising ethanol.

Advantageously, in the above mentioned wetting phase, ethanol is present in a concentration by weight, estimated with respect to the total weight of the leaf fragments of Tamarindus indica L., equal to approximately 20%.

In addition, the process comprises a maceration phase of the leaves of Tamarindus indica L. in an organic solvent to obtain a mixture.

Preferably, the organic solvent consists of ethanol.

In detail, the leaves of Tamarindus indica L. are dipped in a maceration mixture comprising ethanol present in a concentration by weight, estimated with respect to the total weight of the mixture, equal to approximately 72%, and water present in a concentration by weight, estimated with respect to the total weight of the composition, equal to approximately 28%.

In this regard, it is specified that the maceration phase lasts between 48 and 96 hours.

Preferably, the maceration phase lasts approximately 72 hours.

Afterwards, the process comprises an extraction phase of the Tamarind extract from the mixture obtained during the maceration phase to obtain a solution.

The extraction phase comprises a percolation step of the solution obtained in the extraction phase at a speed substantially equal to 5 nal/min.

The Tamarind extract obtained at the end of the percolation step is hereinafter identifiable with the acronym “EBT” (Tamarind soft extract).

The percolation step is carried out by means of a percolator device of a type known to the technician in the field.

At this point, the Tamarind extract thus obtained includes ethanol which must be removed to allow the use thereof in formulations for oral administration. For this purpose, the process comprises a concentration phase of the Tamarind extract.

The concentration phase is carried out by means of a rotary evaporator.

It cannot however be ruled out from the scope of the present treatise that the concentration phase can be carried out using super-critical fluids.

In addition, the above mentioned concentration phase is carried out at an operating temperature substantially equal to 40° C.

At the end of the concentration phase, the ratio of Tamarind extract thus obtained to ethanol is substantially equal to 4:1 (m/v).

In detail, at the end of the concentration phase, the concentrated tamarind extract is obtained, for brevity below indicated with the acronym “EMT”.

In addition, the present invention relates to a Tamarind extract that can be obtained with the previously described process.

The above mentioned extract comprises extracted molecules belonging to:
polyphenols in a concentration by weight, estimated in relation to the total weight of the extract, ranging from 3% to 9%, preferably from 4.26% to 8.12%; and
flavonoids in a concentration by weight, estimated in relation to the total weight of the extract, ranging from 1% to 3%, preferably from 3.08% to 3.32%.

The extract also has a pH comprised between 3 and 5, preferably between 3.08 and 3.32 and a density comprised between 0.7 g/ml and 1.5 g/ml.

Preferably, the extract has a pH of approximately 3.85.

It is specified that the extract according to the present invention has:
color: dark brown;
flavor: acid and astringent;
total solids: present in a concentration by weight, estimated in relation to the total weight of the extract, comprised between 60% and 70%;
total ashes: present in a concentration by weight, estimated in relation to the total weight of the extract, comprised between 1% and 2%.

In addition, this extract is used for the treatment of liver disease, diabetes, hyperlipidemia and oxidative stress disorders.

Finally, the present invention relates to the use of the extract for the preparation of a granular pharmaceutical composition for oral administration.

This pharmaceutical composition is in one of the following forms: liquid, solid and semi-solid.

More specifically, the pharmaceutical composition is in a form selected from the list comprising: syrup, solution, suspension, granule, powder, tablet, capsule, cream, jelly and paste.

It cannot be ruled out from the scope of the present treatise that the pharmaceutical composition may be in the form of nano-capsule, nano-sphere and liposome.

The granular pharmaceutical composition is obtained by means of a wet granulation process of the type known to the technician in the sector.

The granulate obtained this way undergoes a drying phase until a predetermined residual moisture content is obtained.

In detail, the drying phase of the granulate is carried out at a temperature comprised between 35° C. and 45° C.

Preferably, the drying phase of the granulate is carried out at a temperature of approximately 40° C.

Afterwards, the granulate thus obtained has been compressed to 20 MPa by means of an eccentric machine using flat, beveled punches with a diameter of 12.7 mm, to obtain 600 mg tablets.

Below, by way of example, is a pharmaceutical composition comprising the following components, present in a percentage by weight, estimated in relation to the overall weight of the composition:

| | |
|---|---|
| Excipients | 30%-37% |
| Microcrystalline Cellulose | 30%-40% |
| Lactose monohydrate | 4.5%-6.5% |
| Polyvinylpyrrolidone sodium | 3%-5% |
| Aerosil | 1%-5% |
| Magnesium stearate | 1%-2% |
| EBT according to the present invention | 20%. |

Alternative formulations do not rule out the presence of the following components in the relative concentrations by weight, estimated in relation to the overall weight of the composition:

| | |
|---|---|
| Microcrystalline Cellulose | 47.62%-71.73% |
| Corn starch pre-gelatinized | 23.81%-47.62% |
| in situ | |
| Aerosil ® | 5%-5% |
| EBT | 20%-20% |

Antioxidant, Hepatoprotective Activity and Lipid Profile In Vivo

Below are the results of an experiment involving 5 experimental groups of Sprague Dawley rats.

Each group comprises 7 Sprague Dawley rats.

Group I (control): oral administration of carboxymethylcellulose, daily for 7 days, in a concentration by weight of 0.5% in distilled water and in an amount equivalent to 1 ml/100 g of body weight.

Group II (Carbon Tetrachloride Group): intraperitoneal administration of carbon tetrachloride, daily for 7 days, in an amount equivalent to 0.5 nal/kg of body weight. On the second day of experimentation, carbon tetrachloride was administered, intraperitoneally, with olive oil, in a 1:3 ratio.

Group III (silymarin group): oral administration of silymarin suspended in 0.5% carboxymethylcellulose and distilled water at a concentration of 150 mg/kg, daily for 7 days. Silymarin is administered in an amount equivalent to 1 ml per 100 g of body weight.

Group IV (group of 100 mg/kg tablets): administration of the EMT extract in powder form at a concentration of 100 mg/kg total body weight, suspended in 0.5% carboxymethylcellulose and distilled water. The EMT extract is administered in an amount equivalent to 1 ml per 100 g of body weight.

Group V (group of 200 mg/kg tablets): administration of the EMT extract in powder form at a concentration of 200 mg/kg total body weight and suspended in 0.5% carboxymethylcellulose and distilled water. The EMT extract is administered in an amount equivalent to 1 ml per 100 g of body weight.

All groups received water and conventional food for the entire duration of the experimentation. In addition, each animal was fasting 18 hours before being sacrificed with ethyl ether narcosis.

Evaluation of Antioxidant Activity

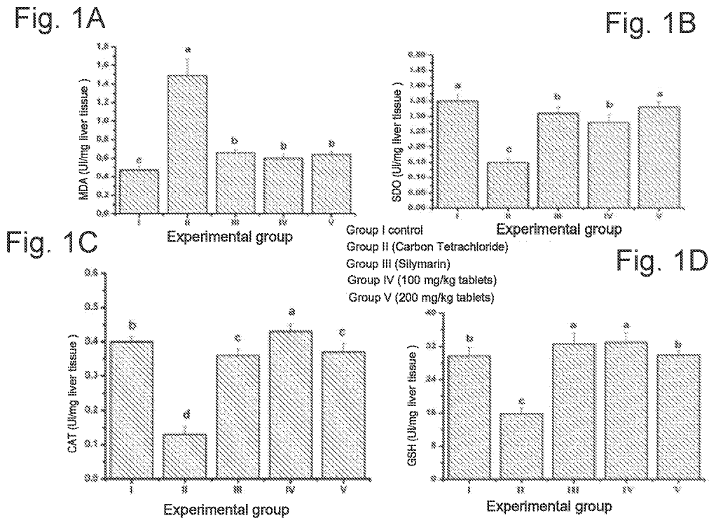

As can be seen in FIG. 1, to assess the antioxidant activity has been determined the tissue concentration of:
Malondialdehyde (MDA) (Reckagel, 1967, Han et al. 2000);
Catalase (CAT) (Aebi, 1984);
Superoxide dismutase (SOD) (Malstron et al., 1975);
Glutathione (GSH) (Akerboom and Sies, 1981);

As visible in FIGS. 1A-1D, the administration of EMT in both doses (Group IV and Group V) shows an unexpected inhibitory activity of lipid peroxidation.

It is clear, therefore, how the synergy of the components present in the EMT extract contributes significantly to increase the antioxidant effect compared to the effect carried out by the individual components separately.

Hepatoprotective Activity

The serum levels (Table 1) of alanine aminotransferase (ALT), aspartate aminotransferase (AST), gamma-glutamyl transferase (GGT), alkaline phosphatase (ALP), total bilirubin (TB) and total protein (TP) were estimated.

The table below shows the values measured for the effect of granulates administered on biochemical markers of the liver function serum.

TABLE 1

| | ALT | AST | ALP | GGT | TB | TP |
|---|---|---|---|---|---|---|
| Group | (UI/L) | (UI/L) | (UI/L) | (UI/L) | (mg/100 mL) | (g/100 mL) |
| I | 32.79 ± 0.31 | 21.53 ± 0.41 | 184.53 ± 0.51 | 89.53 ± 3.31 | 4.09 ± 0.41 | 7.96 ± 0.49 |
| II | 333.54 ± 20.15 | 99.21 ± 22.31 | 423.53 ± 0.51 | 164.35 ± 6.41 | 7.53 ± 0.08 | 4.52 ± 0.34 |
| III | 44.98 ± 0.64 | 46.87 ± 1.26 | 236.56 ± 1.51 | 95.20 ± 1.14 | 7.35 ± 0.30 | 8.43 ± 0.21 |
| IV | 33.21 ± 0.55 | 33.33 ± 1.42 | 203.76 ± 0.65 | 96.13 ± 1.18 | 5.21 ± 0.14 | 10.93 ± 0.45 |
| V | 33.24 ± 1.07 | 33.24 ± 1.07 | 200.51 ± 0.55 | 97.30 ± 3.30 | 5.73 ± 0.07 | 10.68 ± 0.43 |

It is noted that the groups IV and V produced a surprisingly greater effect than silymarin concentrated at 150 mg/kg.

Also in this, the synergy of the components of the EMT extract is easily observed.

Evaluation of the Hypolipidic Activity In Vivo

The table below (Table 2) shows the serum lipidic profile determined for all experimental groups.

Similarly to the previous cases, also in this case the groups IV and V show a more powerful effect compared to silymarin concentrated at 150 mg/kg.

TABLE 2

| | Triglycerides | Cholesterol | HDL-c | LDL-c | VLDL-c |
|---|---|---|---|---|---|
| Group | (mg/100 mL) | (mg/100 mL) | (mg/100 mL) | (mg/100 mL) | (mg/100 mL) |
| I | 51.79 ± 0.57c | 89.88 ± 5.31c | 50.53 ± 1.31c | 36.25 ± 1.31b | 9.01 ± 1.41b |
| II | 123.27 ± 6.15a | 155.21 ± 6.11a | 23.03 ± 2.49a | 104.99 ± 4.51a | 27.43 ± 2.08a |
| III | 57.44 ± 2.21b | 96.01 ± 2.26b | 62.46 ± 2.51b | 34.26 ± 2.13b | 15.35 ± 2.30b |
| IV | 52.00 ± 2.02c | 93.85 ± 2.37c | 51.33 ± 1.25c | 34.77 ± 2.18b | 12.44 ± 2.15b |
| V | 53.29 ± 2.41c | 94.11 ± 1.27c | 52.12 ± 2.35c | 33.26 ± 1.32c | 12.23 ± 0.97b |

Acute Oral Toxicity in Sprague Dawley Rats

The experimental test involved two groups of Sprague Dawley rats comprising 7 rats each. Below are the regimens of administration carried out to both groups:
Group I: Control group—water only was administered;
Group II: Experimental group—a dose of 2000 mg/kg of Tamarindus indica L. (TIL) granules was administered.

Within the above mentioned experimental protocol, no clinical signs have been detected that could be associated with systemic toxic effects caused by TIL granules. Both the appearance of mucous membranes and eyes and somatomotor activity has not changed with respect to before administration.

TABLE 3

| Organ | Group I (%) | Group II (%) | t-test; p-value |
|---|---|---|---|
| Kidneys | 0.80 ± 0.18 | 0.78 ± 0.35 | −0.91; 0.382 |
| Pancreas | 0.38 ± 0.09 | 0.37 ± 0.12 | 1.21; 0.475 |
| Heart | 0.32 ± 0.02 | 0.39 ± 0.33 | 0.41; 0.927 |
| Lungs | 0.60 ± 0.21 | 0.62 ± 0.34 | 2.01; 0.469 |
| Liver | 4.48 ± 0.47 | 4.22 ± 0.11 | 1.28; 0.852 |

Water administration in Group II was 41.2 ml/day and food administration was 20.38 g/day. These values are not statistically different from the amounts of water and food ingested by Group I.

The experimental test ended with a 100% survival and the relative weight of internal organs in both groups showed no significant statistical differences.

Sub-Chronic Oral Toxicity for 28 Days

The experimental test was carried out to evaluate the potential toxic effects of the TIL tablets when administered continuously in a dose of 1000 mg/kg body weight of the animals for 28 days.

Two experimental groups comprising three animals each were involved:
Group I: Control group—only distilled water and food were administered;
Group II: Experimental group—100 mg/kg body weight was administered orally;

In the table below (Table 4) the biochemical parameters are related to the serum of both experimental groups for the evaluation of sub-acute toxicity.

Specifically, this table shows the values for alanine amino transferase (ALT), aspartate aminotransferase (AST), creatinine, urea, alkaline phosphatase (ALP), total protein (TP), cholesterol, triglycerides and glycemia for both groups.

TABLE 4

| Parameters | Group I | Group II | t-test; p-value |
|---|---|---|---|
| ALT (UL-1) | 34.28 ± 1.82 | 36.38 ± 2.11 | 1.45; 0.125 |
| AST (UL-1) | 23.12 ± 1.22 | 24.52 ± 2.32 | 2.01; 0.569 |
| ALP (UL-1) | 7.58 ± 0.28 | 7.11 ± 0.91 | 0.21; 1.523 |
| TP (mg/100 ml) | 5.60 ± 0.41 | 5.01 ± 1.22 | 3.11; 1.697 |
| Urea (mg/100 ml) | 24.35 ± 1.35 | 26.12 ± 2.23 | 2.08; 0.985 |
| Triglycerides | 45.28 ± 1.35 | 44.77 ± 2.85 | 3.21; 0.920 |
| (3 g/100 ml) | | | |
| Cholesterol | 94.59 ± 3.37 | 94.22 ± 1.09 | 1.01; 0.069 |
| (mg/100 ml) | | | |
| Blood Glucose | 104.48 ± 3.97 | 107.13 ± 3.45 | 3.01; 1.539 |
| (mg/100 ml) | | | |

No clinical signs associable with any systemic toxic effects caused by TIL granulate were observed when administered for 28 days.

Similarly, no changes were observed in the mucous membranes, eyes and skin of animals. The experimental test concluded with 100% survival.

Similarly, in the study of biochemical parameters, no statistically significant changes were found in liver function, kidney function, pancreatic function and serum lipid profiles (Table 5).

The relative weight of internal organs for both experimental groups, after administration for 28 days at the dose of 1000 mg/kg showed no statistically significant differences, demonstrating that TIL granulate does not cause damage to the functionality of internal organs.

TABLE 5

| Organ | Group I (%) | Group II (%) | t-test; p-value |
|---|---|---|---|
| Kidneys | 0.77 ± 0.11 | 0.71 ± 0.27 | 2.41; 0.652 |
| Pancreas | 0.33 ± 0.12 | 0.32 ± 0.54 | 1.20; 0.098 |
| Heart | 0.33 ± 0.05 | 0.30 ± 0.99 | 2.21; 0.111 |
| Lungs | 0.55 ± 0.33 | 0.57 ± 0.83 | 2.98; 0.452 |
| Liver | 3.33 ± 0.57 | 3.74 ± 0.43 | 1.88; 0.098 |
| Spleen | 0.30 ± 0.25 | 0.32 ± 0.17 | 1.47; 0.810 |

It has, in practice, been ascertained that the described invention achieves the intended objects.

In particular, it is emphasized that the synergistic presence of flavonoids and polyphenols allows activating the response in the front line of the antioxidant defense system, i.e. the endogenous enzymatic system.

1) Process for the production of Tamarind extract, wherein said process comprises:
a) a supply phase of a vegetable substrate comprising leaves of Tamarindus indica L.;
b) a drying phase of said leaves of Tamarindus indica L.;
c) a grinding phase of said leaves of Tamarindus indica L.;
d) a maceration phase of said leaves of Tamarindus indica L. in an organic solvent to obtain a mixture;
e) an extraction phase of said Tamarind extract from the mixture obtained in d) to obtain a solution; and
f) a concentration phase of said Tamarind extract.

2) Process according to claim 1, wherein said drying phase comprises a shielding step of said leaves of Tamarindus indica L.

3) Process according to claim 1, wherein said grinding phase comprises a sifting step to obtain fragments of said leaves of Tamarindus indica L. ranging from 2.5 mm to 10 mm.

4) Process according to claim 1, wherein said organic solvent comprises ethanol.

5) Process according to claim 3, wherein said process comprises a wetting phase of said fragments of said leaves of Tamarindus indica L. for a period ranging from 1 hour to 5 hours, said wetting phase being prior to said maceration phase.

6) Process according to claim 1, wherein said extraction phase comprises a percolation step of said solution obtained in e) at a speed substantially equal to 5 ml/min.

7) Process according to claim 1, wherein said concentration phase is carried out by means of rotary evaporator.

8) Process according to claim 1, wherein said concentration phase is carried out at an operating temperature substantially equal to 40° C.

9) Process according to claim 1, wherein the ratio of Tamarind extract to ethanol is substantially equal to 4:1 (m/v).

10) Extract obtainable with the process according to claim 1, wherein said extract comprises extracted molecules belonging to:
polyphenols in a concentration by weight, estimated in relation to the total weight of the extract, ranging from 6% to 8%;
flavonoids in a concentration by weight, estimated in relation to the total weight of the extract, ranging from 1% to 3%;
wherein said extract has a pH comprised between 3 and 5 and a density comprised between 0.7 g/ml and 1.5 g/ml.

11) Extract according to claim 10, for use in the treatment of liver disease, diabetes, hyperlipidemia and oxidative stress disorders.

12) Use of the extract according to claim 10 for the preparation of a solid, liquid or semi-solid pharmaceutical composition by oral administration.
